(19) United States
(12) Patent Application Publication
(10) Pub. No.: US 2014/0287995 A1
(43) Pub. Date: Sep. 25, 2014

(54) STABLE PHARMACEUTICAL FORMULATIONS OF GROWTH FACTOR PEPTIDES

(71) Applicant: THE UNIVERSITY OF CHICAGO, Chicago, IL (US)

(72) Inventors: F. Gary Toback, Chicago, IL (US); Ann Berger, Kalamazoo, MI (US)

(21) Appl. No.: 14/354,835

(22) PCT Filed: Oct. 29, 2012
(86) PCT No.: PCT/US2012/062375

§ 371 Date: Apr. 28, 2014

Related U.S. Application Data
(60) Provisional application No. 61/554,575, filed on Nov. 2, 2011.

(51) Int. Cl. A61K 38/10; A61K 47/26; A61K 47/22; A61K 38/08

(52) U.S. Cl. CPC A61K 38/10; A61K 38/08; A61K 47/26; A61K 47/22
USPC 514/7.6

(57) ABSTRACT

The present disclosure provides pharmaceutical formulations including a kidney growth factor peptide and one or more excipients, wherein the formulations have a pH of greater than about 6.8. The disclosure also provides processes for preparing the formulation and products prepared by such processes.

### CROSS REFERENCE TO RELATED APPLICATIONS

This application claims the benefit of priority under 35 U.S.C. §119(e) to U.S. Provisional Patent Application No. 61/554,575, filed Nov. 2, 2011. The disclosures set forth in the referenced application is incorporated herein by reference in its entirety.

The instant application contains a Sequence Listing which has been submitted in ASCII format via EFS-Web and is hereby incorporated by reference in its entirety. Said ASCII copy, created on Oct. 24, 2012, is named 700175_SEQ_ST25.txt and is 8,103 bytes in size.

### BACKGROUND

Growth promoting peptides derived from protein factors advantageously stimulate mitogenic activity of epithelial cells. For example, such peptides have demonstrated stimulation of mitogenic activity in kidney epithelial cells. In particular, peptides designated “kidney growth factor” proteins and peptides due to their original source, have shown the ability to stimulate growth of epithelial cells (e.g., U.S. Pat. No. 6,096,706).

Administration of kidney growth factor proteins and/or peptides to animals provides therapeutic benefits, for example, for the treatment of acute renal failure. Accordingly, a pharmaceutical formulation including kidney growth factor proteins and/or peptides is advantageous to utilize the therapeutic potential of such proteins and peptides. A pharmaceutical formulation ideally sustains the biological activity of the proteins and peptides at a desired level, as well as maintains the stability of the proteins and/or peptides for a desirable period of time.

In the art of protein and peptide formulations, tissue culture assays are generally used for evaluating effectiveness of the protein or peptide, for example evaluation of the biological activity of the formulated proteins and peptides. The tissue culture assays typically utilize conventional growth culture media containing growth factors and nutrients, and the media are usually highly buffered at a neutral pH. Even if a protein or peptide formulation has an acidic pH, combining the formulation with conventional tissue culture media would be expected to neutralize the protein or peptide formulation towards a neutral pH and retain the desired biological activity of the protein or peptide in the formulation.

However, pharmaceutical formulations developed according to conventional methods known in the art can result in decreased biological activity of the active ingredients, for example, kidney growth factor proteins or peptides present in the formulations. Unexpectedly, pharmaceutical formulations developed according to conventional methods and having an acidic pH exhibit decreased biological activity, even when the formulations are combined with conventional tissue culture media. Thus, undesirable pharmaceutical formulations of kidney growth factor proteins or peptides possess unexpectedly decreased biological activity as a result of the procedures utilized during the formulation process. Furthermore, undesirable pharmaceutical formulations of kidney growth factor proteins or peptides can have unfavorable stability properties.

### SUMMARY OF THE DISCLOSURE

Pharmaceutical formulations are disclosed that are suitable for kidney growth factor proteins and/or peptides because they maintain the desired biological activity of the proteins or peptides and also provide related advantages. Unexpectedly, formulations using routine methods and compositions deleteriously affected the biological activity of the peptides, as compared with expectations from results in vitro. Accordingly, the present disclosure provides suitable pharmaceutical formulations.

The present disclosure demonstrates that the decreased biological activity of kidney growth factor proteins or peptides was overcome with a pharmaceutical formulation including certain excipients and utilizing a specified pH. By preparing such a pharmaceutical formulation, kidney growth factor proteins or peptides maintained biological activity and are useful as therapeutic agents for the treatment of disease.

A pharmaceutical formulation is disclosed that includes at least one kidney growth factor peptide, and one or more excipients. The formulation has a pH of greater than about 6.8.

Suitable excipients include sucrose, histidine, their biological equivalents or combinations thereof.

The desired pH range of the formulations is from about 6.8 to about 8.0; or about 7.0 to about 7.5; or about 7.1 to about 7.4; or about 7.2 to about 7.3; or about 7.25-7.50, or about 7.25. The “about” herein takes into account statistical variation based on measurement techniques.

The formulation preferably includes at least one kidney growth factor peptides characterized by the following amino acid sequences:

| | |
|---|---|
| AQPYPQGNHEXXYG | (SEQ ID NO: 1) |
| YPQGNH | (SEQ ID NO: 2) |
| YPQGN | (SEQ ID NO: 3) |
| AQPYPQGNHEATSSSF | (SEQ ID NO: 4) |
| AQPYPQGNHEATSSS | (SEQ ID NO: 5) |
| AQPYPQGNHEA | (SEQ ID NO: 6) |
| AQPYPQGNHEAT | (SEQ ID NO: 7) |
| AQPYPQGNHEATS | (SEQ ID NO: 8) |
| AQPYPQGNHEATSS | (SEQ ID NO: 9) |
| AQPYPQGNHEATSY | (SEQ ID NO: 10) |
| AQPYPQGNHEAAYG | (SEQ ID NO: 11) |
| AQPYPQGNHEAAY | (SEQ ID NO: 12) |
| AQPYPQGNHEAA | (SEQ ID NO: 13) |
| AQPYPQGNHE | (SEQ ID NO: 14) |
| AQPYPQGNHEASYG | (SEQ ID NO: 15) |
| AQPYPQGNHEASY | (SEQ ID NO: 16) |
| AQPYPQGNHEAS | (SEQ ID NO: 17) |
| QPYPQGNHEA | (SEQ ID NO: 18) |
| AQPYPQGNH | (SEQ ID NO: 19) |
| QPYPQGNHE | (SEQ ID NO: 20) |
| PYPQGNHEA | (SEQ ID NO: 21) |
| QPYPQGNH | (SEQ ID NO: 22) |
| PYPQGNHE | (SEQ ID NO: 23) |
| YPQGNHEA | (SEQ ID NO: 24) |
| PYPQGNH | (SEQ ID NO: 25) |
| YPQGNHE | (SEQ ID NO: 26) |
| YPQGNHEATSSSF | (SEQ ID NO: 27) |
| YPQGNHEATSSS | (SEQ ID NO: 28) |
| YPQGNHEATSS | (SEQ ID NO: 29) |
| YPQGNHEATS | (SEQ ID NO: 30) |
| YPQGNHEAT | (SEQ ID NO: 31) |

or biological equivalents. That is, if the sequences include, at either terminus, amino acids or other molecules that do not substantially change the cell growth promoting activity of the peptides, they are within the scope of the claims. If equivalent amino acids replace those in the disclosed sequences, but the cell growth promoting activity is equivalent, those substitutions do not move the disclosed peptides out of the claim scope.

Growth of the cells is meant to include mitogenic stimulation.

A growth factor peptide designated NX001 includes the amino acid sequence AQPYPQGNHEASYG (SEQ ID NO: 15).

Concentration of the kidney growth factor peptide in the formulation disclosed herein is about 0.1 mg/mL to about 100 mg/mL; or about 20 mg/mL; or about 80 mg/mL.

Sucrose is present at a molarity of about 1 mM to about 500 mM; about 15 mM; or about 200 mM.

Histidine is present at a molarity of about 1 mM to about 500 mM; or about 20 mM.

A suitable pH may be obtained by neutralizing the formulation after combining the kidney growth factor peptide and the one or more excipients. A suitable pH may also be obtained by neutralizing the formulation with addition of a pharmaceutically acceptable acid or base.

The synthesized kidney growth factor peptide is prepared via lyophilization.

A process for preparing a kidney growth factor peptide formulation includes combining the kidney growth factor peptide and one or more excipients, and neutralizing the formulation to a pH of greater than about 6.8 subsequent to combination.

Products made from the formulation processes disclosed herein are within the scope of the claims.

### BRIEF DESCRIPTION OF DRAWINGS

Other aspects of the present disclosure will be readily appreciated as the same becomes better understood by reference to the following detailed description when considered in connection with the accompanying drawings which are illustrative, but not limiting.

FIG. 1 shows the mitogenic potency of unformulated NX001 peptide on non-transformed African Green Monkey kidney epithelial (BSC-1) cells. Two lots of unformulated NX001 peptide were compared. Each value is the mean±SE for three separate experiments, each of which was performed in triplicate (N=9 cultures). The arrow indicates a 25% stimulation of cell growth.

FIG. 2 shows the mitogenicity of formulated NX001. Two vials from the same lot are compared. One vial is graphed using open circles, and the other using closed circles. The lot tested is PPL-WGF0801. PPL-WGF0801 (0.125 μg/ml, not formulated) was used as a positive control (closed square). Values are means±SE for 3 separate experiments, each of which was performed in triplicate (N=9 cultures).

FIG. 3 shows the effect of sucrose, histidine, or both on the mitogenicity of formulated NX001. Samples were tested for mitogenicity either alone (Panel A) or with NX001 (Panel B). Panel A: Excipient was diluted with water and an aliquot was added to a culture of BSC-1 cells. Panel B: NX001 (8 μl, 0.125 μg/μl) was added to the culture medium without or with an excipient. A control group (C) received 8 μl of water. The number of cells was counted in a hemocytometer 4 days later. Each value is the mean±SE (N=3 cultures).

FIG. 4 shows the effect of pH on the mitogenicity of formulated NX001. As a negative control (C) 8 μl of water was added to a culture, whereas a positive control (bar 1) received NX001 peptide alone (0.2 μg/ml). For bars 2-4, a solution of 20 mM histidine with 8.8% sucrose pH 7.0 in water was prepared, and split into three aliquots, which were adjusted to pH 5.5 (using 0.1 N HCl), and pH 7.5 and 9.0 with 0.1 N NaOH (bars 2, 3, and 4, respectively). For bars 5-7, NX001 was added to each of these excipient solutions, which resulted in a drop in pH to 5.0, 5.5 and 6.5, respectively, and diluted 1/160 with water to a final concentration of 0.125 μg/μl (bars 5, 6, and 7, respectively). For bars 8-10, 8 μl of each of the 3 of excipient solutions (2-4 above) was added to individual cultures followed by 8 μl of NX001 (0.125 μg/μl) (bars 8, 9, and 10, respectively). For bars 11-13, the set of solutions that contained excipients and NX001 shown in bars 5-7 were each immediately neutralized to pH 7.5 with 0.1 N NaOH, then diluted 1/160 with water (bars 11, 12, and 13, respectively). For bars 14-16 (as controls for bars 11-13), an aliquot of each formulation solution 2-4 was neutralized to pH 7.5 with 0.1 N NaOH and added to a cell monolayer (bars 14, 15, and 16, respectively). Values are means±SE for 3 cultures.

FIG. 5 shows that neutralization of formulated preparations of NX001 restores mitogenesis. Cultures assayed for mitogenic activity in bar C (control) had no additions, whereas water was added to cultures shown in bar 1, and PBS in bar 2. The growth-promoting effect of 0.125 μg/mL NX001 dissolved in water is shown in bar 3 and in PBS in bar 4. NX001 formulated in 20 mM histidine, 8.8% sucrose, (final pH 5.5), diluted to 0.125 μg/mL with water is shown in bar 5. An aliquot of NX001 formulated in 20 mM histidine, 8.8% sucrose, (final pH 5.5) that was neutralized to pH 7.5 with 0.1 N NaOH and then diluted to 0.125 μg/mL with water is shown in bar 6. Another aliquot of formulated NX001 that was diluted in PBS (pH 7.25) to 0.125 μg/mL is depicted in bar 7. Additions (8 μl each) were made to the culture medium and the number of BSC-1 cells in 3 cultures was counted 4 days later. Values are means±SE.

FIG. 6 shows the mitogenicity of neutral NX001 formulations. Values are means±SE for 6 cultures. The arrow indicates NX001-mediated 25% stimulation of cell growth.

### DETAILED DESCRIPTION OF THE DISCLOSURE

The present disclosure provides pharmaceutical formulations including a kidney growth factor peptide and one or more excipients, wherein the formulations have a pH of greater than about 6.8. A range of about 6.8-8.0 is with the scope of the disclosure. The disclosure also provides processes for preparing the formulation and products prepared by such processes.

The pharmaceutical formulations according to the present disclosure provide several advantages compared to formulations developed according to conventional methods known in the art. First, the pharmaceutical formulations of the present disclosure allow for the kidney growth factor peptide in the formulations to maintain biological activity. Second, the pharmaceutical formulations of the present disclosure maintain necessary stability of the peptide in the formulations. Third, the lyophilization process to produce the peptide allows for desirable levels of biological activity and stability of the pharmaceutical formulations. Finally, the process for preparing the pharmaceutical formulations of the present disclosure allows for pH neutralization by a variety of mechanisms in order to maintain biological activity of the peptide in the formulations.

In some embodiments of the present disclosure, a pharmaceutical formulation is described. The pharmaceutical formulation includes a kidney growth factor peptide, one or more excipients, wherein the formulation has a pH of greater than about 6.8. As used herein, a “kidney growth factor peptide” shall include those polypeptides and proteins that have at least one biological activity of stimulating kidney epithelial cell growth, as well as analogs, mutants, pharmaceutically acceptable salts, altered glycosylated peptides, PEG conjugated peptides, isoforms, mimetics, fragments, hybrid proteins, fusion proteins, oligomers and multimers, homologues, glycosylation pattern variants, variants, splice variants, and muteins, thereof, regardless of the biological activity of same, and further regardless of the method of synthesis or manufacture thereof including, but not limited to, recombinant (whether produced from cDNA, genomic DNA, synthetic DNA or other form of nucleic acid), in vitro, in vivo, by microinjection of nucleic acid molecules, synthetic, transgenic, and gene activated methods. Additionally, the term kidney growth factor peptide encompasses kidney growth factor polypeptides including one or more amino acid substitutions, additions, or deletions, with equivalent biological activity. The biological activity of stimulating kidney epithelial cell growth is well known to the skilled artisan.

The term “pharmaceutically acceptable salt” refers to a salt that exists in conjunction with the acidic or basic portion of the kidney growth factor peptide. Such salts include the pharmaceutically acceptable salts listed in HANDBOOK OF PHARMACEUTICAL SALTS: PROPERTIES, SELECTION AND USE, P. H. Stahl and C. G. Wermuth (Eds.), Wiley-VCH, New York, 2002 which are known to the skilled artisan.

In the various illustrative embodiments described herein, a kidney growth factor peptide is characterized by an amino acid sequence selected from the following group:

| | |
|---|---|
| AQPYPQGNHEXXYG | (SEQ ID NO: 1) |
| YPQGNH | (SEQ ID NO: 2) |
| YPQGN | (SEQ ID NO: 3) |
| AQPYPQGNHEATSSSF | (SEQ ID NO: 4) |
| AQPYPQGNHEATSSS | (SEQ ID NO: 5) |
| AQPYPQGNHEA | (SEQ ID NO: 6) |
| AQPYPQGNHEAT | (SEQ ID NO: 7) |
| AQPYPQGNHEATS | (SEQ ID NO: 8) |
| AQPYPQGNHEATSS | (SEQ ID NO: 9) |
| AQPYPQGNHEATSY | (SEQ ID NO: 10) |
| AQPYPQGNHEAAYG | (SEQ ID NO: 11) |
| AQPYPQGNHEAAY | (SEQ ID NO: 12) |
| AQPYPQGNHEAA | (SEQ ID NO: 13) |
| AQPYPQGNHE | (SEQ ID NO: 14) |
| AQPYPQGNHEASYG | (SEQ ID NO: 15) |
| AQPYPQGNHEASY | (SEQ ID NO: 16) |
| AQPYPQGNHEAS | (SEQ ID NO: 17) |
| QPYPQGNHEA | (SEQ ID NO: 18) |
| AQPYPQGNH | (SEQ ID NO: 19) |
| QPYPQGNHE | (SEQ ID NO: 20) |
| PYPQGNHEA | (SEQ ID NO: 21) |
| QPYPQGNH | (SEQ ID NO: 22) |
| PYPQGNHE | (SEQ ID NO: 23) |
| YPQGNHEA | (SEQ ID NO: 24) |
| PYPQGNH | (SEQ ID NO: 25) |
| YPQGNHE | (SEQ ID NO: 26) |
| YPQGNHEATSSSF | (SEQ ID NO: 27) |
| YPQGNHEATSSS | (SEQ ID NO: 28) |
| YPQGNHEATSS | (SEQ ID NO: 29) |
| YPQGNHEATS | (SEQ ID NO: 30) |
| YPQGNHEAT | (SEQ ID NO: 31) |

In one illustrative embodiment described herein, a kidney growth factor peptide is characterized by an amino acid sequence AQPYPQGNHEASYG (SEQ ID NO: 15).

The pharmaceutical formulations of the present disclosure utilize various excipients. Sucrose, histidine, citric acid, percholoric acid, sodium citrate, sodium perchlorate, mannitol, and trehalose, or any combination thereof, can be used as excipients according to the pharmaceutical formulations of the present disclosure. Other pharmaceutically acceptable excipients known to those practiced in the art are also suitable. In some embodiments of the present disclosure, an excipient is sucrose. In some embodiments, sucrose has a molarity of about 1 mM to about 500 mM. In other embodiments, sucrose has a molarity of about 1 mM to about 250 mM. In other embodiments, sucrose has a molarity of about 1 mM to about 100 mM. In yet other embodiments, sucrose has a molarity of about 1 mM to about 50 mM. In other embodiments, sucrose has a molarity of about 10 mM to about 25 mM. In some embodiments, sucrose has a molarity of about 10 mM. In some embodiments, sucrose has a molarity of about 15 mM. In some embodiments, sucrose has a molarity of about 20 mM. In some embodiments, sucrose has a molarity of about 25 mM. In some embodiments, sucrose has a molarity of about 50 mM. In some embodiments, sucrose has a molarity of about 100 mM.

In some embodiments of the present disclosure, an excipient is histidine. In some embodiments, histidine has a molarity of about 1 mM to about 500 mM. In other embodiments, histidine has a molarity of about 1 mM to about 250 mM. In other embodiments, histidine has a molarity of about 1 mM to about 100 mM. In yet other embodiments, histidine has a molarity of about 1 mM to about 50 mM. In other embodiments, histidine has a molarity of about 10 mM to about 25 mM. In some embodiments, histidine has a molarity of about 10 mM. In some embodiments, histidine has a molarity of about 15 mM. In some embodiments, histidine has a molarity of about 20 mM. In some embodiments, histidine has a molarity of about 25 mM. In some embodiments, histidine has a molarity of about 50 mM. In some embodiments, histidine has a molarity of about 100 mM.

The pharmaceutical formulations of the present disclosure have a pH of greater than about 6.8. In some embodiments, the pH of the formulation is from about 6.8 to about 8.0. In some embodiments, the pH of the formulation is from about 7.0 to about 7.5. In some embodiments, the pH of the formulation is from about 7.1 to about 7.4. In some embodiments, the pH of the formulation is from about 7.2 to about 7.3. In some embodiments, the pH of the formulation is about 7.25 to 7.50. In some embodiments the formulation is about 7.25.

The amount of the kidney growth factor peptide in the pharmaceutical formulations is adequate to achieve a therapeutic effect. As used herein, the term “therapeutically effective amount” refers to an amount which gives the desired benefit to an animal and includes both treatment and prophylactic administration. The amount will vary from one individual to another and will depend upon a number of factors, including the overall physical condition of the patient and the underlying cause of the condition to be treated. The amount of kidney growth factor peptide used for therapy gives an acceptable rate of change and maintains desired response at a beneficial level in animals, such as humans.

A therapeutically effective amount of the present compositions may be readily ascertained by one of ordinary skill in the art using publicly available materials and procedures. For example, the amount of the kidney growth factor peptide can be present in the formulation in an amount of between about 0.1 mg/mL to about 100 mg/mL. In some embodiments, the kidney growth factor peptide is present at a concentration of about 1 mg/mL. In some embodiments, the kidney growth factor peptide is present at a concentration of about 10 mg/mL. In some embodiments, the kidney growth factor peptide is present at a concentration of about 20 mg/mL. In some embodiments, the kidney growth factor peptide is present at a concentration of about 25 mg/mL. In some embodiments, the kidney growth factor peptide is present at a concentration of about 30 mg/mL. In some embodiments, the kidney growth factor peptide is present at a concentration of about 50 mg/mL. In some embodiments, the kidney growth factor peptide is present at a concentration of about 75 mg/mL. In some embodiments, the kidney growth factor peptide is present at a concentration of about 80 mg/mL. In some embodiments, the kidney growth factor peptide is present at a concentration of about 85 mg/mL. In some embodiments, the kidney growth factor peptide is present at a concentration of about 90 mg/mL. In some embodiments, the kidney growth factor peptide is present at a concentration of about 100 mg/mL.

In various embodiments of the present disclosure, the pharmaceutical formulations have a pH that is obtained by neutralization of the formulations. As used herein, the term “neutralization” means making a composition to have a more neutral pH (i.e., changing the pH of a composition to a pH of about 6.8 to about 7.5). For example, addition of an acidic substance to a basic composition can make the basic composition to have a more neutral pH (i.e., approximately 6.8-7.5). Likewise, addition of a basic substance to an acidic composition can make the acidic composition to have a more neutral pH (i.e., approximately 6.8-7.5).

In some embodiments of the present disclosure, the pH of the pharmaceutical formulations is obtained by neutralizing the formulation after combining the kidney growth factor peptide and the one or more excipients. In one embodiment, the neutralization is achieved by addition of the kidney growth factor peptide. In another embodiment, the neutralization is achieved by addition of a pharmaceutically acceptable acid or base. In one embodiment, the pharmaceutically acceptable acid or base is sodium hydroxide. In another embodiment, the pharmaceutically acceptable acid or base is sodium acetate. In yet another embodiment, the pharmaceutically acceptable acid or base is sodium citrate. In another embodiment, the pharmaceutically acceptable acid or base is sodium benzoate.

In various embodiments of the present disclosure, the kidney growth factor peptide is prepared via lyophilization. The term “lyophilization,” also known as freeze-drying, is a commonly employed technique for presenting proteins which serves to remove water from the protein preparation of interest. Lyophilization is a process by which the material to be dried is first frozen and then the ice or frozen solvent is removed by sublimation in a vacuum environment.

In other embodiments of the present disclosure, a process for preparing a kidney growth factor peptide formulation is described. The process includes the step of combining kidney growth factor peptide and one or more excipients, wherein the formulation is neutralized to a pH of greater than about 6.8, wherein a range of 6.8-8.0 is acceptable, subsequent to the combination. The previously described embodiments of the pharmaceutical formulations, including excipients, range of pH and specific pHs, and neutralization techniques are applicable to the process of preparing the formulations.

In other embodiments of the present disclosure, a product made by the process for preparing a kidney growth factor peptide formulation is described. The product can be made by the process that includes the step of combining kidney growth factor peptide and one or more excipients, wherein the formulation is neutralized to a pH of greater than about 6.8, wherein a range of 6.8-8.0 is acceptable, subsequent to combination. The previously described embodiments of the pharmaceutical formulations, including excipients, range of pH and specific pHs, and neutralization techniques are applicable to the product made by the process of preparing the formulations.

According to the present disclosure, a formulation containing a kidney growth factor peptide may be administered by any conventional route suitable for proteins or peptides, including, but not limited to, parenterally, e.g. injections including, but not limited to, subcutaneously or intravenously or any other form of injections or infusions. Formulations containing a kidney growth factor peptide can be administered by a number of routes including, but not limited to oral, intravenous, intraperitoneal, intramuscular, transdermal, subcutaneous, topical, sublingual, intravascular, intramammary, or rectal means. Formulations containing a kidney growth factor peptide can also be administered via liposomes. Such administration routes and appropriate formulations are generally known to those of skill in the art. Formulations containing a kidney growth factor peptide, alone or in combination with other suitable components, can also be made into aerosol formulations (i.e., they can be “nebulized”) to be administered via inhalation. Aerosol formulations can be placed into pressurized acceptable propellants, such as dichlorodifluoromethane, propane, nitrogen, and the like.

Formulations containing a kidney growth factor peptide suitable for parenteral administration (e.g., administration via intraarticular, intravenous, intramuscular, intradermal, intraperitoneal, and subcutaneous routes) include aqueous and non-aqueous, isotonic sterile injection solutions (which can contain antioxidants, buffers, bacteriostats, and solutes that render the formulation isotonic with the blood of the intended recipient), and aqueous and non-aqueous sterile suspensions (that can include suspending agents, solubilizers, thickening agents, stabilizers, and preservatives). The formulations containing a kidney growth factor peptide can be presented in unit-dose or multi-dose sealed containers, such as ampules and vials. The formulations containing a kidney growth factor peptide can also be presented in syringes, such as prefilled syringes.

### EXAMPLES

Examples are provided for illustrative purposes and are not intended to limit the scope of the disclosure.

### Example 1

Various lots of unformulated NX001 can be assayed for bioactivity using the mitogenic response of BSC-1 cells as an indicator (see, for instance, U.S. Pat. No. 6,096,706). In this example, two lots of NX001 peptide (Lots PPL-WGF0501A and lot PPL WGF0801) were tested. Varying amounts of the two peptides were added to near-confluent monolayers of non-transformed African Green Monkey kidney epithelial (BSC-1) cells, and the number of cells was counted in a hemocytometer 4 days later.

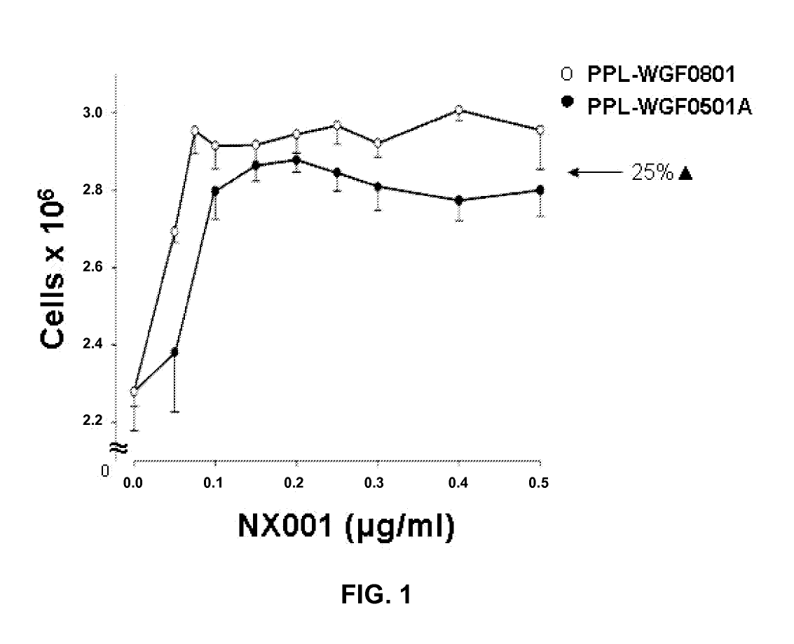

The two lots of unformulated NX001 peptide have similar concentration-dependent mitogenic profiles when assayed in monolayer cultures of non-transformed monkey kidney epithelial cells of the BSC-1 line (see FIG. 1). The ED₅₀ for PPL-WGF0801 was 0.038 μg/mL and the ED₅₀ for PPL-WGF0501A was 0.069 μg/mL (no significant difference). Thus, both lots of NX001 peptide had similar maximal mitogenic responses (˜26%) when tested in vitro.

### Example 2

To test the bioactivity of formulated and lyophilized NX001, replicates of formulation samples were assayed. Lyophilized formulations of NX001 lots PP-WGF0801 were dissolved at 20 mg/mL in 20 mM histidine, 254 mM sucrose, pH 6.5. Then, 1 mL aliquots were lyophilized in 3 mL vials.

Each formulation was reconstituted with 1.0 mL water, and was swirled to dissolve the contents. Each solution was then be diluted to 0.125 mg/mL with water. Aliquots of 8 μL each was added to triplicate wells of BSC-1 cells to yield final concentrations of NX001 from 0.05 to 0.4 μg/mL. The plates were incubated at 37° C. for 4 days, and then cells was counted. A third lot of NX001 (PPL-WGF0801) that is not formulated and not lyophilized was dissolved and diluted in water and was used as positive control.

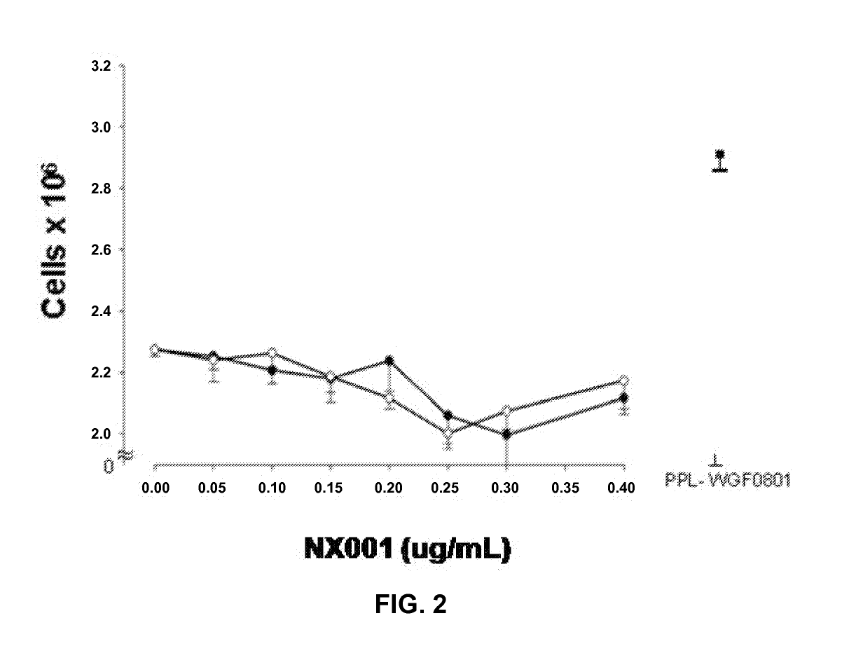

FIG. 2 shows the results of an assay for mitogenicity of the samples. Replicate vials of reconstituted PPL-WGF0801 were not mitogenic compared to the positive control of NX001 that was not formulated and lyophilized (FIG. 2, closed square). Thus, NX001 peptide formulated and lyophilized according to standard procedures may demonstrate a lack of biological activity.

### Example 3

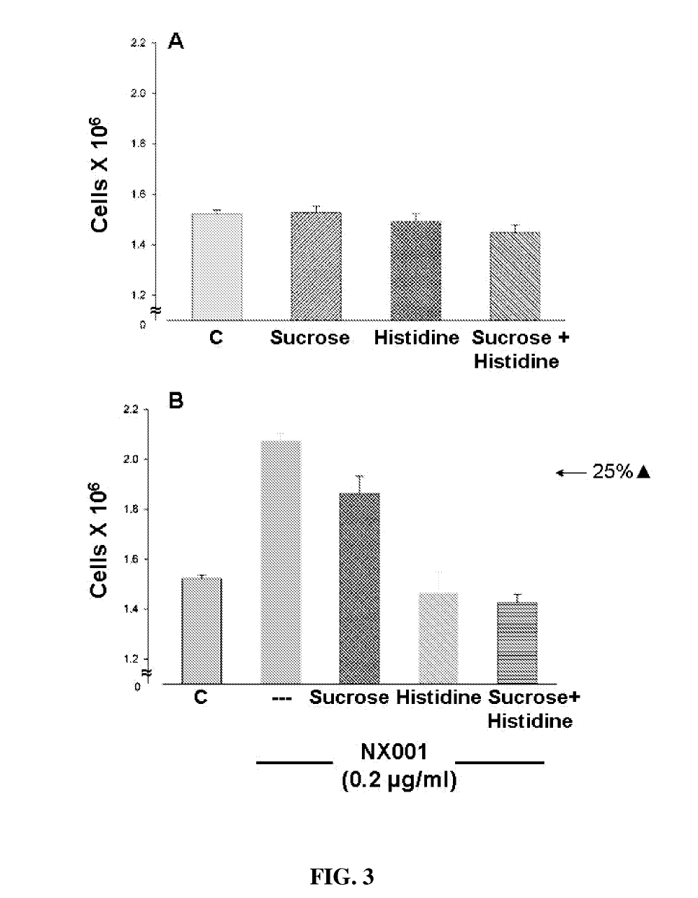

To investigate whether the excipients used in the disclosed formulation was responsible for the unexpected and variable biological activity of the peptide, histidine, sucrose or a combination of histidine and sucrose, was tested for mitogenicity in the presence or absence of NX001. Formulations containing 20 mM histidine, 8.8% sucrose or a combination of the two excipients were prepared, and were diluted 160-fold before addition to the assay. FIG. 3A shows that histidine or sucrose, alone or together, did not exhibit mitogenic activity.

To investigate whether excipients alone or in combination might modify the mitogenic activity, NX001 was added to 20 mM histidine, 8.8% sucrose or the combination and the solutions were diluted to 0.125 mg/mL. Each of the near confluent monolayers of BSC-1 cells in 5 ml DMEM containing 40 μM biotin and 0.5% calf serum received either 8 μl of water, 8 μl of an excipient, or 0.2 μg/ml of PPL-WGF0801 in a final volume of 8 μl of 8.8% sucrose or 20 mM histidine (pH 5.5), or both. FIG. 3B shows that NX001 alone (36% increase in cell number) or in the presence of sucrose (23% increase in cell number) is mitogenic, but that NX001 in the presence of histidine alone or histidine plus sucrose abolished its growth-promoting activity.

Thus, histidine, sucrose, or histidine plus sucrose do not alter growth of BSC-1 cells. However, histidine alone or in the presence of sucrose blocks the mitogenic effect of NX001, whereas sucrose alone does not.

### Example 4

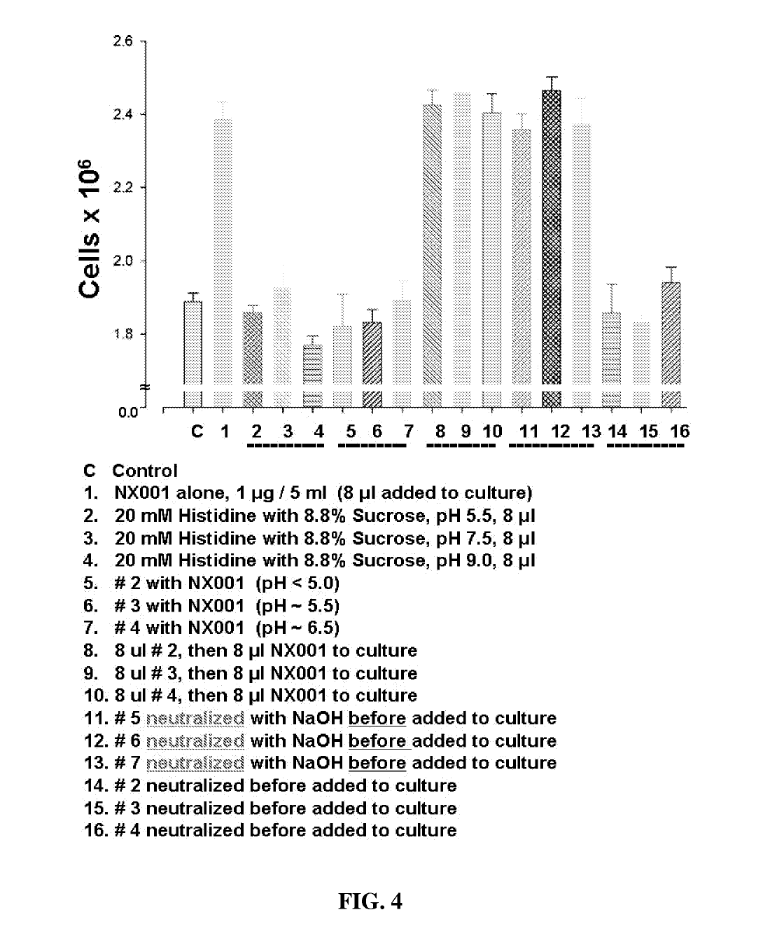

An experiment to determine whether the pH of formulated NX001 affected bioactivity was performed. In this experiment, NX001 (lot PPLWGF0801) was used as a positive control and was dissolved in water to 2 mg/mL, diluted 1/160 with water to 0.125 mg/mL, and 8 μL was added to assay plates to a final concentration of 0.2 μg/mL. (see FIG. 4, bar 1).

Next, 20 mM histidine, containing 8.8% sucrose was prepared and the pH was adjusted to 5.5 with HCl. Similarly, additional solutions of 20 mM histidine containing 8.8% sucrose were prepared and the pH was adjusted to 7.5 and 9.0. These solutions were diluted 1/160 and 8 μL aliquots added to plates of BSC-1 cells.

As shown in FIG. 4 (bars 2, 3, 4), the solutions were not mitogenic themselves. NX001 was dissolved in each of solution to 0.125 mg/mL. Surprisingly, the pH of each solution measured to be <5.0, ˜5.5 and ˜6.5, respectively, suggesting that the addition of NX001 peptide to histidine/sucrose solutions lowers the pH of the solutions.

Each of these solutions was added to culture plates of BSC-1 cells. As shown in FIG. 4 (bars 5, 6, 7), the solutions of NX001 were not mitogenic. Thus, formulations of NX001 prepared with histidine and sucrose excipients and with pH values of 6.5 or lower did not demonstrate biological activity of NX001 when added to tissue culture medium.

### Example 5

The 8 μL of solutions of histidine/sucrose at pHs 5.5, 7.5 and 9.0 were added to plates of BSC-1 cells. NX001 serving as a positive control peptide (8 μl/dish, 0.2 μg/mL medium) were added in a separate addition to the plates.

As shown in FIG. 4 (bars 8, 9, 10), NX001 increased cell growth to the same extent as the positive control alone. Therefore, adding NX001 to the assay in a separate addition from histidine and sucrose resulted in a biological active NX001.

### Example 6

Solutions of NX001 (0.125 μg/mL) in 20 mM histidine, 8.8% sucrose at pHs <5.0, ˜5.5 and ˜6.5 (the same solutions tested in FIG. 4, bars 2, 3, 4) were neutralized with NaOH to a final pH of 7.5 before dilution in water and subsequent addition to assay plates.

As shown in FIG. 4 (bars 11, 12, 13), the solutions, when neutralized, were mitogenic to the same extent as the positive control. Therefore, formulations of NX001 in histidine/sucrose with low pHs retained biological activity if the formulations were neutralized before dilution and addition to the assay. However, formulations of NX001 were inactive if the formulations remained at a pH 6.5 or lower during dilution and addition to the assay.

### Example 7

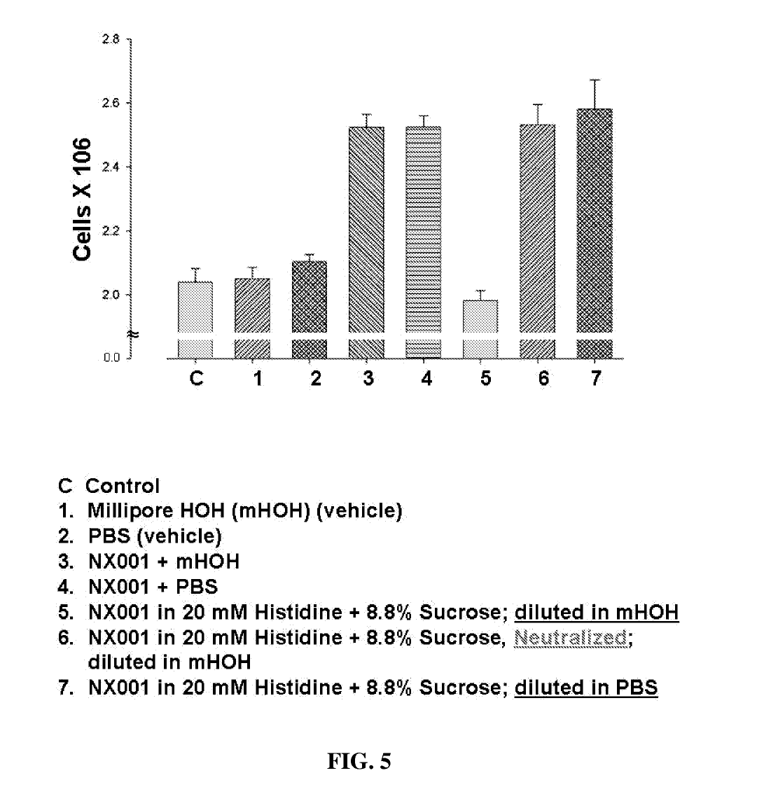

In the present example, controls comprising no addition of peptide, 8 μL water, or phosphate buffered saline (pH 7.5) were added to assay plates and resulted in no increase in cell number (see FIG. 5, bars C, 1, and 2, respectively). NX001 (lot PPLWGF0801) was dissolved to 20 mg/mL in either water or PBS, diluted to 0.125 mg/mL in water or PBS as specified, and 8 μL was then added to assay plates to a final concentration of 0.125 μg/mL. FIG. 5 (bar 3) shows that NX001 dissolved and diluted in water increased the growth of BSC-1 cells by 24%.

FIG. 5 (bar 4) shows that NX001 dissolved in PBS and diluted in water increased cell number by the same amount. As shown in FIG. 5 (bars 5 and 6), NX001 dissolved in histidine/sucrose and diluted in water is inactive (bar 5), whereas NX001 dissolved in histidine/sucrose, neutralized with NaOH, and then diluted in water is active (bar 6). In addition, FIG. 5 (bar 7) shows that if NX001 was dissolved in histidine/sucrose and diluted in PBS before addition to the assay, a >24% increase in cell number was observed.

### Example 8

To insure that formulation of NX001 yielded a solution for injection that was as physiological as possible, the formulation of NX001 was adjusted to provide a neutral, iso-osmotic preparation. The adjusted formulation contains 20 mg/mL NX001, 20 mM histidine, 200 mM sucrose at a pH of 7.25, or 80 mg/mL NX001, 20 mM histidine, 15 mM sucrose, pH 7.25. Vials of formulated and lyophilized NX001 at either 80 mg/mL or 20 mg/mL were reconstituted with 1 mL water and diluted to 0.125 μg/mL before bioassay.

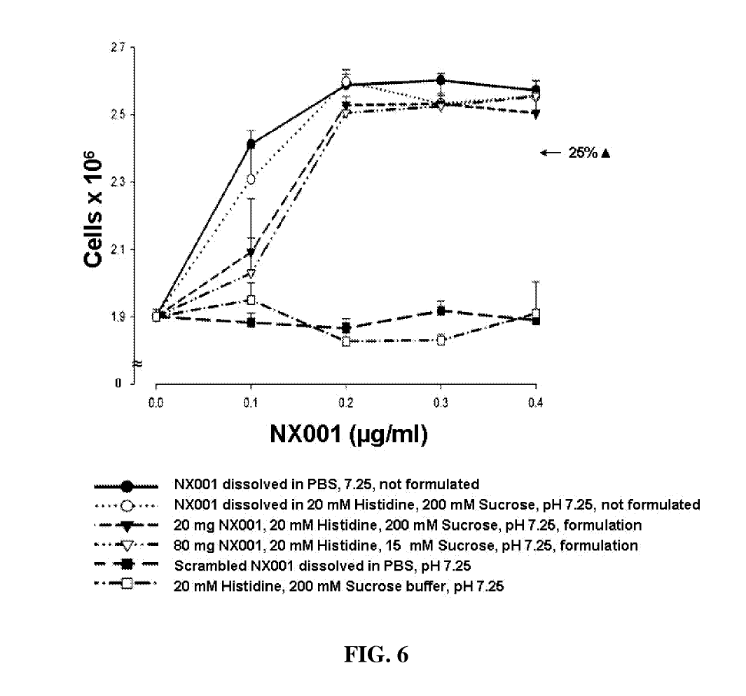

The results of this assay are presented in FIG. 6. NX001 (lot PPL-WGF0501A) dissolved in PBS immediately before assay was active (closed circles), resulting in ˜30% increase in cell number. NX001 (lot PPL-WGF0501A) dissolved in 20 mM histidine, 200 mM sucrose, pH 7.25 immediately before assay was also active as expected (open circles). Scrambled NX001 peptide (gly-tyr-glu-ser-pro-ala-his-gly-tyr-gln-ala-pro-asn-gln (SEQ ID NO: 32), a 14-amino acid peptide including the same amino acids as NX001, but in “scrambled” order, did not promote cell growth (closed squares), nor did 20 mM histidine, 100 mM sucrose, pH 7.25 (open squares).

Formulated, lyophilized, and reconstituted NX001 (20 mg/mL formulation, closed triangles; 80 mg/mL formulation, open triangles) were both active, stimulating a ˜30% increase in cell number. Neither the addition of the stabilizers present in the formulations nor the scrambled peptide had any growth-promoting activity when added to monolayers of BSC-1 cells.

### Example 9

The stability of NX001 formulations was also evaluated. The stability of a lot of NX001 was monitored for 12 months at storage temperatures of 5° C. and 25° C. Appearance of lyophilized cake, reconstitution time, appearance of the reconstituted solution, pH, identity of the peptide, potency and purity of the peptide, and residual moisture were evaluated.

Table 1 shows stability parameters of neutral formulations of NX001. The evaluated parameters of neutral formulations of NX001 were not changed following 12 months storage at temperatures of 5° C. and 25° C.

TABLE 1

| Storage | | | After 12 months |
|---|---|---|---|
| Temperature | Test | Initial | of storage |
| 5° C. | Cake appearance | White cake | White cake |
| | Reconstitution time | 45 seconds | 64 seconds |
| | Reconstituted | Clear, colorless | Clear, colorless |
| | appearance | solution, | solution, |
| | | essentially free of | essentially free of |
| | | particulate matter | particulate matter |
| | pH | 7.2 | 7.2 |
| | Identity¹ | Positive | Positive |
| | Potency² | 102% | 99.6% |
| | Purity | 98.8% | 98.8% |
| | Total % impurities | 1.2% | 1.2% |
| | Residual moisture | 0.71% | 0.80% |
| 25° C. | Cake appearance | White cake | White cake |
| | Reconstitution time | 45 seconds | 72 seconds |
| | Reconstituted | Clear, colorless | Clear, colorless |
| | appearance | solution, | solution, |
| | | essentially free of | essentially free of |
| | | particulate matter | particulate matter |
| | pH | 7.2 | 7.3 |
| | Identity¹ | Positive | Positive |
| | Potency² | 102% | 97.9% |
| | Purity | 98.8% | 98.4% |
| | Total % impurities | 1.2% | 1.6% |
| | Residual moisture | 0.71% | 1.05% |

¹Identity is correspondence of reverse-phase HPLC peak of sample as compared to standard.
²Potency is % of NX001 recovered in main peak compared to stated vial concentration.

1. A pharmaceutical formulation comprising a kidney growth factor peptide and one or more excipients, wherein the formulation has a pH of greater than about 6.8.

2. The formulation of claim 1 wherein at least one excipient is sucrose.

3. The formulation of claim 1 wherein at least one excipient is histidine.

4. The formulation of claim 1 wherein the excipients comprise sucrose and histidine.

5. The formulation of claim 4 wherein the pH is from about 6.8 to about 8.0.

6. The formulation of claim 4 wherein the pH is from about 7.0 to about 7.5.

7. The formulation of claim 4 wherein the pH is from about 7.1 to about 7.4.

8. The formulation of claim 4 wherein the pH is from about 7.2 to about 7.3.

9. The formulation of claim 4 wherein the pH is about 7.25.

10. The formulation of claim 1 wherein the kidney growth factor peptide is selected from the group consisting of:

| | |
|---|---|
| AQPYPQGNHEXXYG, | (SEQ ID NO: 1) |
| YPQGNH, | (SEQ ID NO: 2) |
| YPQGN, | (SEQ ID NO: 3) |
| AQPYPQGNHEATSSSF, | (SEQ ID NO: 4) |
| AQPYPQGNHEATSSS, | (SEQ ID NO: 5) |
| AQPYPQGNHEA, | (SEQ ID NO: 6) |
| AQPYPQGNHEAT, | (SEQ ID NO: 7) |
| AQPYPQGNHEATS, | (SEQ ID NO: 8) |
| AQPYPQGNHEATSS, | (SEQ ID NO: 9) |
| AQPYPQGNHEATSY, | (SEQ ID NO: 10) |
| AQPYPQGNHEAAYG, | (SEQ ID NO: 11) |
| AQPYPQGNHEAAY, | (SEQ ID NO: 12) |
| AQPYPQGNHEAA, | (SEQ ID NO: 13) |
| AQPYPQGNHE, | (SEQ ID NO: 14) |
| AQPYPQGNHEASYG, | (SEQ ID NO: 15) |
| AQPYPQGNHEASY, | (SEQ ID NO: 16) |
| AQPYPQGNHEAS, | (SEQ ID NO: 17) |
| QPYPQGNHEA, | (SEQ ID NO: 18) |
| AQPYPQGNH, | (SEQ ID NO: 19) |
| QPYPQGNHE, | (SEQ ID NO: 20) |
| PYPQGNHEA, | (SEQ ID NO: 21) |
| QPYPQGNH, | (SEQ ID NO: 22) |
| PYPQGNHE, | (SEQ ID NO: 23) |
| YPQGNHEA, | (SEQ ID NO: 24) |
| PYPQGNH, | (SEQ ID NO: 25) |
| YPQGNHE, | (SEQ ID NO: 26) |
| YPQGNHEATSSSF, | (SEQ ID NO: 27) |
| YPQGNHEATSSS, | (SEQ ID NO: 28) |
| YPQGNHEATSS, | (SEQ ID NO: 29) |
| YPQGNHEATS, | (SEQ ID NO: 30) |
| and | |
| YPQGNHEAT. | (SEQ ID NO: 31) |

11. The formulation of claim 4 wherein the kidney growth factor peptide is AQPYPQGNHEASYG (SEQ ID NO: 15).

12. The formulation of claim 4 wherein the kidney growth factor peptide is present at a concentration of about 0.1 mg/mL to about 100 mg/mL.

13. The formulation of claim 4 wherein the kidney growth factor peptide is present at a concentration of about 20 mg/mL.

14. The formulation of claim 4 wherein the kidney growth factor peptide is present at a concentration of about 80 mg/mL.

15. The formulation of claim 11 wherein sucrose is present at a molarity of about 1 mM to about 500 mM.

16. The formulation of claim 11 wherein sucrose is present at a molarity of about 15 mM.

17. The formulation of claim 11 wherein sucrose is present at a molarity of about 200 mM.

18. The formulation of claim 11 wherein histidine is present at a molarity of about 1 mM to about 500 mM.

19. The formulation of claim 11 wherein histidine is present at a molarity of about 20 mM.

20. The formulation of claim 1 wherein the pH is obtained by neutralizing the formulation after combining the kidney growth factor peptide and the one or more excipients.

21. The formulation of claim 20 wherein the neutralization is achieved by addition of the kidney growth factor peptide.

22. The formulation of claim 20 wherein the neutralization is achieved by addition of sodium hydroxide.

23. The formulation of claim 20 wherein the kidney growth factor peptide is prepared via lyophilization.

24. A process for preparing a kidney growth factor peptide formulation, the process comprising the step of combining the kidney growth factor peptide and one or more excipients, wherein the formulation is neutralized to a pH of greater than about 7.0 subsequent to combination.

25. The process of claim 24 wherein the neutralization is achieved by addition of sodium hydroxide.

26. The process of claim 24 wherein the neutralization is achieved by addition of the kidney growth factor peptide.

27. The process of claim 24 wherein the kidney growth factor peptide is selected from the group consisting of:

| | |
|---|---|
| AQPYPQGNHEXXYG, | (SEQ ID NO: 1) |
| YPQGNH, | (SEQ ID NO: 2) |
| YPQGN, | (SEQ ID NO: 3) |
| AQPYPQGNHEATSSSF, | (SEQ ID NO: 4) |
| AQPYPQGNHEATSSS, | (SEQ ID NO: 5) |
| AQPYPQGNHEA, | (SEQ ID NO: 6) |
| AQPYPQGNHEAT, | (SEQ ID NO: 7) |
| AQPYPQGNHEATS, | (SEQ ID NO: 8) |
| AQPYPQGNHEATSS, | (SEQ ID NO: 9) |
| AQPYPQGNHEATSY, | (SEQ ID NO: 10) |
| AQPYPQGNHEAAYG, | (SEQ ID NO: 11) |
| AQPYPQGNHEAAY, | (SEQ ID NO: 12) |
| AQPYPQGNHEAA, | (SEQ ID NO: 13) |
| AQPYPQGNHE, | (SEQ ID NO: 14) |
| AQPYPQGNHEASYG, | (SEQ ID NO: 15) |
| AQPYPQGNHEASY, | (SEQ ID NO: 16) |
| AQPYPQGNHEAS, | (SEQ ID NO: 17) |
| QPYPQGNHEA, | (SEQ ID NO: 18) |
| AQPYPQGNH, | (SEQ ID NO: 19) |
| QPYPQGNHE, | (SEQ ID NO: 20) |
| PYPQGNHEA, | (SEQ ID NO: 21) |
| QPYPQGNH, | (SEQ ID NO: 22) |
| PYPQGNHE, | (SEQ ID NO: 23) |
| YPQGNHEA, | (SEQ ID NO: 24) |
| PYPQGNH, | (SEQ ID NO: 25) |
| YPQGNHE, | (SEQ ID NO: 26) |
| YPQGNHEATSSSF, | (SEQ ID NO: 27) |
| YPQGNHEATSSS, | (SEQ ID NO: 28) |
| YPQGNHEATSS, | (SEQ ID NO: 29) |
| YPQGNHEATS, | (SEQ ID NO: 30) |
| and | |
| YPQGNHEAT. | (SEQ ID NO: 31) |

28. The process of claim 24 wherein the kidney growth factor peptide is AQPYPQGNHEASYG (SEQ ID NO: 15).

29. The process of claim 24 wherein the kidney growth factor peptide is prepared via lyophilization.

30. A product made from the process of claim 24.


SEQUENCE LISTING (32 sequences)

SEQ ID NO: 1 - protein, 14 aa, Artificial Sequence
  Description of Artificial Sequence Synthetic peptide
AQPYPQGNHEXXYG

SEQ ID NO: 2 - protein, 6 aa, Artificial Sequence
  Description of Artificial Sequence Synthetic peptide
YPQGNH

SEQ ID NO: 3 - protein, 5 aa, Artificial Sequence
  Description of Artificial Sequence Synthetic peptide
YPQGN

SEQ ID NO: 4 - protein, 16 aa, Artificial Sequence
  Description of Artificial Sequence Synthetic peptide
AQPYPQGNHEATSSSF

SEQ ID NO: 5 - protein, 15 aa, Artificial Sequence
  Description of Artificial Sequence Synthetic peptide
AQPYPQGNHEATSSS

SEQ ID NO: 6 - protein, 11 aa, Artificial Sequence
  Description of Artificial Sequence Synthetic peptide
AQPYPQGNHEA

SEQ ID NO: 7 - protein, 12 aa, Artificial Sequence
  Description of Artificial Sequence Synthetic peptide
AQPYPQGNHEAT

SEQ ID NO: 8 - protein, 13 aa, Artificial Sequence
  Description of Artificial Sequence Synthetic peptide
AQPYPQGNHEATS

SEQ ID NO: 9 - protein, 14 aa, Artificial Sequence
  Description of Artificial Sequence Synthetic peptide
AQPYPQGNHEATSS

SEQ ID NO: 10 - protein, 14 aa, Artificial Sequence
  Description of Artificial Sequence Synthetic peptide
AQPYPQGNHEATSY

SEQ ID NO: 11 - protein, 14 aa, Artificial Sequence
  Description of Artificial Sequence Synthetic peptide
AQPYPQGNHEAAYG

SEQ ID NO: 12 - protein, 13 aa, Artificial Sequence
  Description of Artificial Sequence Synthetic peptide
AQPYPQGNHEAAY

SEQ ID NO: 13 - protein, 12 aa, Artificial Sequence
  Description of Artificial Sequence Synthetic peptide
AQPYPQGNHEAA

SEQ ID NO: 14 - protein, 10 aa, Artificial Sequence
  Description of Artificial Sequence Synthetic peptide
AQPYPQGNHE

SEQ ID NO: 15 - protein, 14 aa, Artificial Sequence
  Description of Artificial Sequence Synthetic peptide
AQPYPQGNHEASYG

SEQ ID NO: 16 - protein, 13 aa, Artificial Sequence
  Description of Artificial Sequence Synthetic peptide
AQPYPQGNHEASY

SEQ ID NO: 17 - protein, 12 aa, Artificial Sequence
  Description of Artificial Sequence Synthetic peptide
AQPYPQGNHEAS

SEQ ID NO: 18 - protein, 10 aa, Artificial Sequence
  Description of Artificial Sequence Synthetic peptide
QPYPQGNHEA

SEQ ID NO: 19 - protein, 9 aa, Artificial Sequence
  Description of Artificial Sequence Synthetic peptide
AQPYPQGNH

SEQ ID NO: 20 - protein, 9 aa, Artificial Sequence
  Description of Artificial Sequence Synthetic peptide
QPYPQGNHE

SEQ ID NO: 21 - protein, 9 aa, Artificial Sequence
  Description of Artificial Sequence Synthetic peptide
PYPQGNHEA

SEQ ID NO: 22 - protein, 8 aa, Artificial Sequence
  Description of Artificial Sequence Synthetic peptide
QPYPQGNH

SEQ ID NO: 23 - protein, 8 aa, Artificial Sequence
  Description of Artificial Sequence Synthetic peptide
PYPQGNHE

SEQ ID NO: 24 - protein, 8 aa, Artificial Sequence
  Description of Artificial Sequence Synthetic peptide
YPQGNHEA

SEQ ID NO: 25 - protein, 7 aa, Artificial Sequence
  Description of Artificial Sequence Synthetic peptide
PYPQGNH

SEQ ID NO: 26 - protein, 7 aa, Artificial Sequence
  Description of Artificial Sequence Synthetic peptide
YPQGNHE

SEQ ID NO: 27 - protein, 13 aa, Artificial Sequence
  Description of Artificial Sequence Synthetic peptide
YPQGNHEATSSSF

SEQ ID NO: 28 - protein, 12 aa, Artificial Sequence
  Description of Artificial Sequence Synthetic peptide
YPQGNHEATSSS

SEQ ID NO: 29 - protein, 11 aa, Artificial Sequence
  Description of Artificial Sequence Synthetic peptide
YPQGNHEATSS

SEQ ID NO: 30 - protein, 10 aa, Artificial Sequence
  Description of Artificial Sequence Synthetic peptide
YPQGNHEATS

SEQ ID NO: 31 - protein, 9 aa, Artificial Sequence
  Description of Artificial Sequence Synthetic peptide
YPQGNHEAT

SEQ ID NO: 32 - protein, 14 aa, Artificial Sequence
  Description of Artificial Sequence Synthetic scrambled peptide
GYESPAHGYQAPNQ